(19) United States
(12) Patent Application Publication
(10) Pub. No.: US 2022/0248998 A1
(43) Pub. Date: Aug. 11, 2022

(54) SMART MUSCLE TESTING DEVICES AND METHODS FOR USE THEREOF

(71) Applicants: University Of Florida Research Foundation,Incorporated, Gainesville, FL (US); Research Institute at Nationwide Children's Hospital, Columbus, OH (US)

(72) Inventors: Jennifer L. Lammers, Gainesville, FL (US); Lindsay N. Alfano, Dublin, OH (US)

(21) Appl. No.: 17/650,273

(22) Filed: Feb. 8, 2022

Related U.S. Application Data
(60) Provisional application No. 63/147,526, filed on Feb. 9, 2021.

(51) Int. Cl. A61B 5/22; A61B 5/00; A61B 5/107; G16H 40/63

(52) U.S. Cl. CPC A61B 5/224; A61B 5/706; A61B 5/1071; A61B 5/225; G16H 40/63; A61B 2562/0247; A61B 5/702

(57) ABSTRACT

Various embodiments provide a muscle testing device. In an example embodiment, the muscle testing device comprises a frame comprising a central frame element and two arms extending therefrom. The distance between the arms is adjustable. The muscle testing device further comprises a first sensing pad (a) configured to be secured to a first arm of the two arms, (b) configured to measure a force or pressure applied to a sensing surface thereof, and (c) being moveable between a first position and a second position. When the first sensing pad is secured to the first arm in the first position, the sensing surface faces a second arm of the two arms. When the first sensing pad is secured to the first arm in the second position, the sensing surface faces away from the second arm. The muscle testing device may be part of a muscle testing kit.

### CROSS-REFERENCE TO RELATED APPLICATIONS

This application claims priority to U.S. Application No. 63/147,526, filed Feb. 9, 2021, the content of which is incorporated herein by reference in its entirety.

### TECHNICAL FIELD

Various embodiments described herein relate to devices for performing muscle strength testing. For example, smart muscle testing devices and methods for use of the smart muscle testing devices to test muscle strength are described herein.

### BACKGROUND

Muscle strength testing may be used to track the progression of a disease in a patient, determine the effectiveness of a treatment for treating a patient, select assistive devices that are appropriate for a patient, and/or the like. However, conventional methods for muscle strength testing are lacking in accuracy and reproducibility. For example, a clinician may hold a handheld myometer in one hand and use their body to brace the patient in a particular position in order to use the myometer to measure the patient's muscle strength. However, this requires the clinician to assert a resistive force with their hand so as to hold the myometer still while the patient applies a force to the myometer. This leads to a large amount of noise and, ultimately, inaccuracy in the muscle strength testing results.

### BRIEF SUMMARY

Various embodiments described herein are directed to smart muscle testing devices and/or a muscle testing kit. In an example embodiment, the smart muscle testing device is configured to precisely measure muscle strength bilaterally. In an example embodiment, the device includes two sensing pads each selectively secured to an adjustable frame. In various embodiments, the adjustable frame comprises two arms that each extend outward from a central frame element. Each of the arms and the central frame element are configured to be adjusted in (effective) length. For example, the length of the central frame element may be adjusted such that the distance between the opposing sensing pads may be changed. For example, the length of the arms may be adjusted such that the distance between each sensing pad and the central frame element may be changed.

Each of the sensing pads has a force and/or pressure sensor therein and/or a sensor device comprising a force and/or pressure sensor coupled thereto. For example, the sensing pads may comprise a smart sensor material capable of sensing pressure on a surface of the respective sensing pad. For example, the sensing pads may comprise and/or be coupled to a respective force transducer. Thus, the sensing pads are configured to measure a force or pressure applied thereto.

The sensing pads may be moved between a first position and a second position with respect to the arms to which they are secured. In the first position, the respective sensing surfaces of the sensing pads face out from their respective arms. For example, if the sensing pads are both in the first position, the respective sensing surfaces of the sensing pads face away from each other. In the second position, the respective sensing surfaces of the sensing pads face inward from the respective arm (e.g., toward the other arm). For example, if the sensing pads are both in the second position, the respective sensing surfaces of the sensing pads face each other. In an example embodiment, a sensing pad may be rotated about an axis transverse to the central frame element (e.g., an axis defined by the respective arm) to change and/or move the sensing pad from the first position to the second position or vice versa. In an example embodiment, a sensing pad may be detached from the respective arm and then reattached back onto the respective arm to switch the sensing pad between the first position and the second position or vice versa. The sensing pads may be locked into a position along the arm such that they do not move with respect to the respective arm when pressure is applied to the sensing pads during a muscle test.

The muscle testing device may further comprise a user interface configured to provide the results of a muscle test in a human perceivable manner and/or a wired or wireless communications interface for providing the results of a muscle test (e.g., to a computing entity).

According to a first aspect, a muscle testing device is provided. In an example embodiment, the muscle testing device comprises a frame comprising a central frame element and two arms extending from the central frame element. A distance between the two arms is adjustable. The muscle testing device further comprises a first sensing pad (a) configured to be secured to a first arm of the two arms, (b) configured to measure a force or pressure applied to a sensing surface thereof, and (c) being moveable between a first position about the first arm and a second position about the first arm.

In an example embodiment, when the first sensing pad is secured to the first arm in the first position, the sensing surface of the first sensing pad faces a second arm of the two arms and when the first sensing pad is secured to the first arm in the second position, the sensing surface of the first sensing pad faces away from the second arm.

In an example embodiment, the first sensing pad is moveable between the first position and the second position by rotating a pad bracket configured to couple the at least one sensor pad to the first arm with respect to the first arm.

In an example embodiment, the first sensing pad comprises a cradle and a sensor device, the cradle is coupled to the first arm by a pad bracket, and the cradle is configured to couple the sensor device to the first arm, the sensor device comprising the sensing surface.

In an example embodiment, the first sending pad comprises a cradle that couplable to the first arm and configured to couple a sensor device comprising the sensing surface to the first arm.

In an example embodiment, the cradle comprises a sensor receptacle configured to receive the sensor device at least partially therein.

In an example embodiment, the pad bracket is configured to couple the cradle to the first arm at a selected position along the first arm such that the first arm has a selected effective length.

In an example embodiment, the cradle and the pad bracket are configured such that the cradle is rotatable with respect to the pad bracket, while the cradle is coupled to the pad bracket, about an axis that is transverse to a direction substantially parallel to the first arm.

In an example embodiment, the cradle and the sensor device are configured such that the sensor device is rotatable with respect to the cradle, while the sensor device is coupled to the cradle, about the axis that is transverse to the direction substantially parallel to the first arm.

In an example embodiment, the sensor device is a myometer.

In an example embodiment, the muscle testing device further comprises at least one user interface configured to provide human perceivable output of a muscle strength measurement.

In an example embodiment, the user interface is integrated into the at least one sensing pad.

In an example embodiment, the muscle testing device further comprises a pressure and/or force sensor integrated into the at least one sensing pad and comprising the sensing surface, the pressure and/or force sensor configured to measure pressure and/or force applied to the sensing surface thereof.

In an example embodiment, the first arm and second arm are respectively movable between respective in-use positions where a respective arm of the first arm and the second arm extends in a direction that is transverse to a direction substantially parallel to the central frame element and respective stowed positions where the respective arm extends in a direction that is substantially parallel to the central frame element.

In an example embodiment, the muscle testing device further comprises a second sensing pad or a support pad coupled to the second arm.

According to another aspect, a muscle testing kit is provided. In an example embodiment, the muscle testing kit comprises at least one of a muscle testing device or a muscle testing device frame. The muscle testing device comprises a frame comprising a central frame element and two arms extending from the central frame element, wherein a distance between the two arms is adjustable; and a first sensing pad (a) configured to be secured to a first arm of the two arms, (b) configured to measure a force or pressure applied to a sensing surface thereof, and (c) being moveable between a first position and a second position. When the first sensing pad is secured to the first arm in the first position, the sensing surface of the first sensing pad faces a second arm of the two arms and when the first sensing pad is secured to the first arm in the second position, the sensing surface of the first sensing pad faces away from the second arm. the muscle testing device frame comprises the frame comprising the central frame element and the two arms extending from the central frame element, wherein the distance between the two arms is adjustable; and a cradle coupled to the first arm of the two arms via a pad bracket and configured to couple a sensor device to the first arm, wherein when the cradle is secured to the first arm in the first position, a sensing surface of the sensor device coupled to the cradle faces the second arm and when the cradle is secured to the first arm in the second position, the sensing surface of the sensor device coupled to the cradle faces away from the second arm.

In an example embodiment, the muscle testing kit comprises a stand, the at least one of the muscle testing device or the muscle testing device frame configured to be coupled to the stand and the stand configured to maintain the at least one of the muscle testing device or the muscle testing device frame in one or more designated configurations when the at least one of the muscle testing device or the muscle testing device frame is coupled to the stand.

In an example embodiment, the muscle testing kit comprises an alignment mat illustrating positioning of one or more limbs of a patient for one or more muscle testing measurements.

In an example embodiment, the alignment mat is a jig board configured to have the at least one of the muscle testing device or the muscle testing device frame coupled thereto in one or more designated configurations for the one or more muscle testing measurements.

In an example embodiment, the muscle testing kit comprises a table or plinth attachment configured to secure the at least one of the muscle testing device or the muscle testing device frame to a table or plinth.

In an example embodiment, the muscle testing kit further comprises a grip attachment configured to measure grip strength.

In an example embodiment, the muscle testing kit comprises one or more support pads configured to be selectively secured to the at least one of the muscle testing device or the muscle testing device frame and to be positioned against a patient's body to properly position the patient's body for one or more muscle testing measurements.

In an example embodiment, the muscle testing kit is configured to enable muscle testing measurements of one or more of ankle dorsiflexion, ankle plantarflexion, elbow flexion, elbow extension, functional elbow flexion, functional elbow extension, knee flexion, knee extension, hip abduction, hip adduction, hip extension supine, hip abduction clamshell, shoulder abduction, shoulder adduction, shoulder horizontal adduction, shoulder internal rotation, shoulder external rotation, grip strength, neck lateral flexion, neck extension, and neck forward flexion.

According to another aspect, an ankle range of motion measuring device is provided. In an example embodiment, the ankle range of motion measuring device comprises a first planar member; a second planar member; and a goniometer. The first planar member comprises a first surface. The second planar member comprises a second surface. The first surface of the first planar member is coupled to the second surface of the second planar member in a hinged manner. The goniometer is coupled to the first and second planar members such that the goniometer is configured to measure an angle between the first planar member and the second planar member.

In an example embodiment, the angle range of motion measuring device further comprises a first strap configured to strap a human foot to the first planar member; and a second strap configured to strap a human leg to the second planar member.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1A is a side view of a muscle testing device having sensing pads oriented in a first position, in accordance with an example embodiment;

FIG. 1B is a side view of the muscle testing device shown in FIG. 1A and having the sensing pads oriented in a second position, in accordance with an example embodiment;

FIG. 2 is a sideview of a muscle testing device, in accordance with another example embodiment;

FIG. 3 is a side view of a sensing pad, in accordance with an example embodiment;

FIG. 4 is a side view of a sensing pad, in accordance with another example embodiment;

FIG. 5A is a back perspective view of a cradle configured to secure a sensing pad to a muscle testing device, according to an example embodiment;

FIG. 5B is a front perspective view of the cradle shown in FIG. 5A, in accordance with an example embodiment;

FIG. 6 is a perspective view of an example cradle mounting bracket, in accordance with an example embodiment;

FIG. 7 is a top view of an example alignment mat, in accordance with an example embodiment;

FIG. 8 is a side view of an example stand, in accordance with an example embodiment;

FIG. 9 is a side view of a table or plinth attachment securing a muscle testing device or muscle testing device frame to a table or plinth, in accordance with an example embodiment;

FIG. 10 is side view of muscle testing device frame having support pads secured thereto and a grip attachment coupled thereto, in accordance with an example embodiment; and

FIG. 11 is an example ankle range of motion device, in accordance with an example embodiment.

### DETAILED DESCRIPTION

Example embodiments will now be described more fully hereinafter with reference to the accompanying drawings, in which some, but not all embodiments are shown. Indeed, various embodiments may be embodied in many different forms and should not be construed as limited to the embodiments set forth herein; rather, these embodiments are provided so that this disclosure will satisfy applicable legal requirements. Like numbers refer to like elements throughout.

### Example Muscle Testing Device

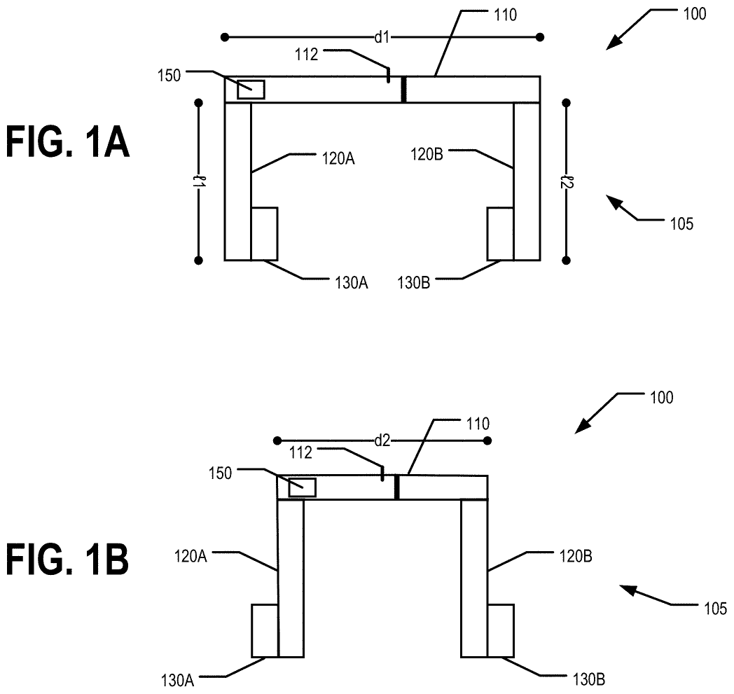

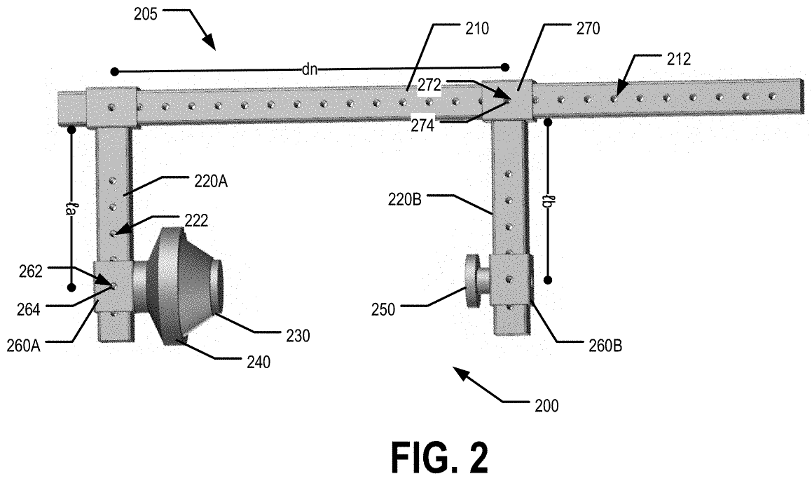

FIGS. 1A and 1B illustrate an example embodiment of a muscle testing device 100 in two different configurations. FIG. 2 illustrates another example embodiment of a muscle testing device 200. In various embodiments, the muscle testing device 100, 200 comprises a frame 105, 205 and one or more sensing pads 130 (e.g., 130A, 130B), 230 configured to be secured thereto. In various embodiments, the frame 105, 205 comprises a central frame element 110, 210, a first arm 120A, 220A, and a second arm 120B, 220B. The first arm 120A, 220A and the second arm 120B, 220B each extend outwardly from the central frame element 110, 210. In various embodiments, a sensing pad 130A, 130B, 230 is secured and/or coupled to a respective arm 120, 200. In the embodiment illustrated in FIGS. 1A and 1B, a sensing pad 130A, 130B is secured and/or coupled to each arm 120A, 120B. In various embodiments, the frame 105, 205 of the muscle testing device 100, 200 is generally shaped as three sides of a rectangle or U-shaped.

In an example embodiment, one or more of the arms 120A, 120B, 220A, 220B are moveable and/or foldable between an in-use position and a stowed position. When in the in-use position, the arms 120A, 120B, 220A, 220B extend outward from and/or extend in a direction transverse to the central frame element 110, 210, as shown in FIGS. 1A, 1B, and 2. When in the stowed position, the arms 120A, 120B, 220A, 220B extend substantially parallel to the central frame element 110, 210. In an example embodiment, this enables a muscle testing device 100, 200 to be stowed (e.g., in the stowed position) in a carrying bag or case or a storage cabinet. The arms 120A, 120B, 220A, 220B of the muscle testing device 100, 200 may then be moved and/or unfolded such that the muscle testing device 100, 200 and/or the frame/arms thereof is in the in-use position for use in performing one or more measurements.

In various embodiments, the central frame element 110 has an adjustable length. For example, in FIG. 1A, the central frame element 110 is configured with a length d1, while in FIG. 1B the central frame element 110 has been adjusted to have a length d2 (which is less than d1). The first arm 120A has a length [symbol] 1 and the second arm 120B has a length [symbol] 2. In an example embodiment, the lengths [symbol] 1 and [symbol] 2 are independently adjustable. In various embodiments, the central frame element 110, first arm 120A, and second arm 120B may be lockable such that a length of the central frame element 110, first arm 120A, and/or second arm 120B is not adjustable while a measurement is being performed.

For example, in the embodiment illustrated in FIGS. 1A and 1B, the central frame element 110 (and/or the first arm 120A and/or the second arm 120B) may comprise two components that are configured to selectively move with respect to one another and then be secured in relationship to one another by a fixing element 112 to adjust the length thereof. For example, the central frame element 110 (and/or the first arm 120A and/or the second arm 120B) may comprise telescoping tubes and the fixing element 112 may comprise a fixing pin configured to enable the telescoping tubes to selectively move with respect to one another and otherwise to be in a fixed relationship to adjust the length of the central frame element 110 (and/or the first arm 120A and/or the second arm 120B). For example, the central frame element 110 (and/or the first arm 120A and/or the second arm 120B) may comprise two or more substantially flat pieces and the fixing element 112 may comprise a bolt or pin component configured to enable the substantially flat pieces to selectively move with respect to one another and otherwise to be in a fixed relationship to adjust the length of the central frame element 110 (and/or the first arm 120A and/or the second arm 120B). In an example embodiment, the length of the central frame element 110 is not adjustable and the arms 120A, 120B are secured to the central frame element 110 such that at least one of the arms 120A, 120B may be slid along at least a portion of the central frame element 110 and secured into one or more positions thereon such that the distance between the arms 120A, 120B is adjustable. In an example embodiment, the arms 120A, 120B are fixed to the central frame element 110 in a fixed (e.g., non-moveable manner, such as welding and/or the like) and the length of the central frame element 110 is adjustable.

In the example embodiment illustrated in FIG. 2, the length of the arms 220A, 220B and/or central frame element 210 are not adjustable. For example, a sensing pad 230 and/or support pad 250 may be secured at various positions along the arms 220A, 220B rather than adjusting the length of the arms. For example, the muscle testing device 200, shown in FIG. 2, is configured such that the effective lengths [symbol] a and [symbol] b of arms 220A, 220B are adjustable while the lengths of the arms 220A, 220B themselves are static. In the illustrated embodiment of FIG. 2, the muscle testing device 200 is configured such that the effective length do of the central frame element 210 is adjustable while the length of the central frame element remains constant.

For example, in the embodiment illustrated in FIG. 2, each arm 220A, 220B includes and/or defines a number of predefined pad attachment locations 222. For example, the predefined pad attachment locations 222 comprise holes (e.g., through holes) and/or other bracket securing components that enable the pad bracket 260A, 260B to be secured to the arm 220A, 220B at the respective predefined pad attachment locations 222. For example a pad bracket 260A, 260B comprises a bracket attachment mechanism 262. In an example embodiment, the bracket attachment mechanism is a hole (e.g., a through hole) and/or other mechanism configured to mate with and/or be coupled to respective predefined pad attachment location 222. In the illustrated embodiment where the predefined pad attachment locations 222 and the bracket attachment mechanism 262 are both through holes, a pin or other mechanical fastener 264 extends through the respective predefined pad attachment location 222 and the bracket attachment mechanism 262 so as to secure the pad bracket 260A at the respective predefined pad attachment location 222. For example, a clinician or other user may secure the pad bracket 260A to a particular predefined pad attachment location 222 by positioning the pin or other mechanical fastener 264 through the bracket attachment mechanism 262 and the particular predefined pad attachment location 222.

Similarly, the central frame component 210 comprises and/or defines a number of predefined arm attachment locations 212. For example, the predefined arm attachment locations 212 comprise holes (e.g., through holes) and/or other bracket securing components that enable the frame bracket 270 to be secured to the central frame element 210 at the respective predefined arm attachment locations 212. For example an frame bracket 270 comprises a bracket attachment mechanism 272. In an example embodiment, the bracket attachment mechanism 272 is a hole (e.g., a through hole) and/or other mechanism configured to mate with and/or be coupled to respective predefined arm attachment location 212. In the illustrated embodiment where the predefined pad attachment locations 212 and the bracket attachment mechanism 272 are both through holes, a pin or other mechanical fastener 274 extends through the respective predefined pad attachment location 212 and the bracket attachment mechanism 272 so as to secure the frame bracket 270 at the respective predefined arm attachment location 212. For example, a clinician or other user may secure the frame bracket 270 to a particular predefined arm attachment location 212 by positioning the pin or other mechanical fastener 274 through the bracket attachment mechanism 272 and the particular predefined pad attachment location 212. In an example embodiment, a pad bracket 260 may be attached, coupled, and/or secured to a predefined arm attachment location 212, as appropriate for one or more measurements to be performed.

In various embodiments, the predefined pad attachment locations 222 and/or predefined arm attachment locations 212 are respectively spaced apart from one another by a respective attachment spacing. In various embodiments, the respective attachment spacing is quarter of an inch, half an inch, an inch, and/or the like, such that effective lengths of the central frame element do and/or the effective length of the arms [symbol] a and [symbol] b can be modified with an appropriate level of precision.

In an example embodiment, the central frame element 210, first arm 220A, second arm 220B, and/or sensing pad 230 are configured such that the effective length of the arms 220A, 220B and/or central frame element 210 are static/constant and/or not adjustable while a measurement is being performed.

In an example embodiment, each arm 120A, 120B may be rotated around its attachment point and locked in position in various embodiments to provide stabilization of the frame during muscle testing. In an example embodiment, frame brackets 270 and/or pad brackets 260A, 260B may be secured to the central frame element 210 and/or a respective arm 220A, 220B to stabilize and/or lock the muscle testing device 200 into a particular position and/or configuration for use in capturing one or more measurements. In various embodiments, each arm 120A, 120B, 220A, 220B can be rotated independently and can be locked into a user-selected position. In various embodiments, a plurality of positions are predefined and a clinician or other user may rotate a respective arm 120A, 120B, 220A, 220B and lock the respective arm into a selected one of the plurality of positions. For example, the positions may be predefined by the formation of a through hole for a fixing pin to be inserted therethrough, other mating and/or securing mechanisms disposed appropriately to define the predefined positions, markings on the respective arm 120A, 120B, 220A, 220B, frame bracket 270, and/or the like. For example, in various embodiments, the arms 120A, 120B, 220A, 220B are selectively rotatable about respective axes extending along the respective arms and transverse to the central frame element 110, 210. In various embodiments, the predefined positions are defined and/or configured to enable the securing of a sensing pad 130, 230, and/or support pad 250 with appropriate precision for the application.

In various embodiments, the frame 105, 205 may be made out of a light-weight, high-strength, high-stiffness material. For example, the frame 105, 205 (e.g., the central frame element 110, 210, the first arm 120A, 220a, and/or the second arm 120B, 220B) may be made out of stainless steel, aluminum, carbon fiber composite, and/or the like. In various embodiments, the frame 105, 205 is made out of a material configured to withstand the stresses and torques applied thereto when forces or pressures within the sensible range of the sensing components of the sensing pads 130A, 130B, 230.

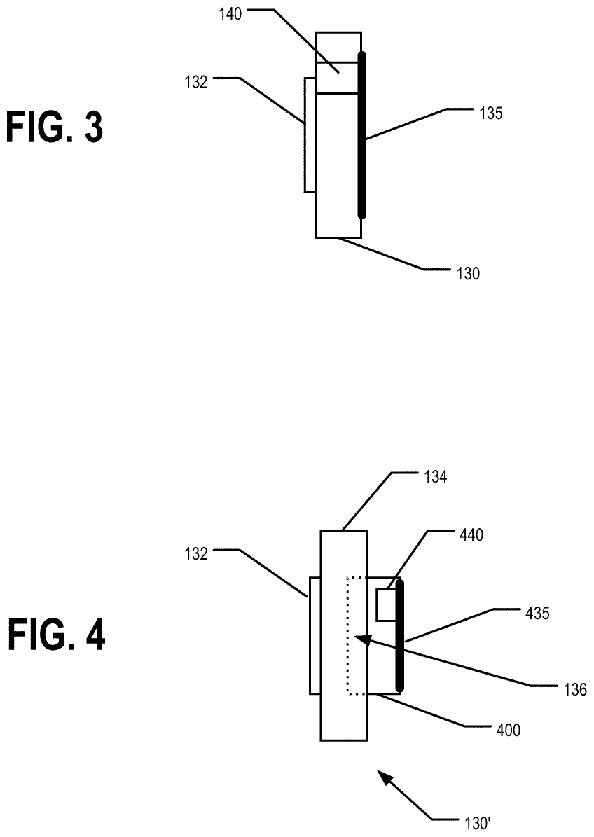

In various embodiments, the muscle testing device 100, 200 further comprises at least one sensing pad 130A, 130B, 230 configured to be secured to the frame 105, 205. For example, the first sensing pad 130A, 230 may be secured to the first arm 120A, 220A and/or the second sensing pad 130B may be secured to the second arm 120B. In an example embodiment, the muscle testing device 200 comprises a support pad 250 secured to one of the arms 220 and/or the central frame element 210. In various embodiments, the first and second sensing pads 130A, 130B are adjustable between a first position (shown in FIG. 1A) and a second position (shown in FIG. 1B). FIG. 2 illustrates an example embodiment of a muscle testing device 200 including one sensing pad 230 secured to a first arm 220A and one support pad 250 secured to a second arm 220B, with the sensing pad 230 and the support pad 250 both arranged in the first position. As shown in FIGS. 1A and 2, when the sensing pads 130A, 130B, 230 (and/or support pad 250) are secured to their corresponding arms 120A, 120B, 220A, 220B in the first position, the sensing surface 135, 435 (see FIGS. 3 and 4) of the sensing pad 130A, 130B, 230 (or the supporting surface of the support pad 250) faces inward (e.g., toward the other arm). As shown in FIG. 1B, when the sensing pad 130A, 130B, 230 is secured to the corresponding arm 120A, 120B, 220A, 220B in the second position, the sensing surface 135, 435 of the sensing pad 130A, 130B, 230 faces outward (e.g., away from the other arm). In various embodiments, each sensing pad 130A, 130B, 230 and/or each support pad 250 are each independently moveable between the first position and the second position.

In various embodiments, the sensing pads 130A, 130B, 230 and/or support pads 250 are each independently moveable between the first position and the second position. In various embodiments, a sensing pad 130, 230 and/or support pad 250 is moveable between the first position and the second position (or vice versa) by rotating the respective pad about an axis that is substantially defined by the respective arm 120, 220. In an example embodiment, a sensing pad 130, 230 and/or support pad 250 is moveable between the first position and the second position (or vice versa) by rotating about a respective arm 120, 220. For example, the pad bracket 260 is configured to rotate about a respective arm 220, in an example embodiment. For example, the pad bracket 260 is removable from the respective arm 220 and then may be repositioned and/or secured to the arm 220 in a rotated manner, in an example embodiment. In an example embodiment, the first and second arms 120A, 120B, 220A, 220B are configured to rotate about an axis defined by the respective arm (and transverse and/or substantially perpendicular to an axis defined by the central frame element 110, 210) such that a sensing pad 130, 230 and/or support pad 250 coupled to a respective arm are rotated between the first and second positions by rotation of the respective arm. For example, an arm 120, 220 (i.e., one or both of the arms 120A, 120B, 220A, 220B) may be secured to the central frame element 110, 210 such that the arm 120, 220 may be selectively rotated about an axis that is defined by the arm 120, 220 and that is transverse to the central frame element 110, 210. For example, the arm bracket 270 is configured to enable rotation of an arm 220 about an axis defined by the arm 220 and that is transverse to the central frame element 210 in an example embodiment. In various embodiments, the sensing pads 130, 230, support pads 250 and/or the first and second arms 120A, 120B, 220A, 220B are able to be locked into position (e.g., so that they cannot rotate with respect to the central frame element 110) such that the sensing pads 130, 230 and/or support pads 250 are retained in a respective one of the first position or the second position while performing a measurement.

In various embodiments, the first position and the second position are two examples of a plurality of positions at which the sensing pads 130, 230 and/or support pads 250 may be secured. For example, in various embodiments, three, four, or more positions may be predefined at which the sensing pads 130, 230 and/or support pads 250 may be secured. For example, in various embodiments, a sensing pad 130, 230 and/or support pad 250 can be rotated about a respective arm 120, 220 and can be locked into a user-selected position. In various embodiments, a plurality of positions are predefined and a clinician or other user may rotate the sensing pad 130, 230, and/or support pad 250 with respect to a respective arm 120, 220 and lock the sensing pad 130, 230, and/or support pad 250 into a selected one of the plurality of positions. For example, the positions may be predefined by the formation of a through hole for a fixing pin to be inserted therethrough, other mating and/or securing mechanisms disposed appropriately to define the predefined positions, markings on the respective arm 120, 220, pad bracket 260, and/or the like. In various embodiments, the predefined positions are defined and/or configured to enable the securing of a sensing pad 130, 230, and/or support pad 250 with appropriate precision for the application.

In various embodiments, the positions at which the sensing pads 130, 230 and/or support pads 250 may be secured include positions where the sensing surface of the sensing pad 130, 230 and/or a supporting surface of the support pad is facing toward the central frame element 110, 210, away from the central frame element 110, 210, and/or an angle therebetween (e.g., toward the opposing arm, away from the opposing arm, at a 60 degree angle with respect to the arm to which the sensing pad and/or support pad is coupled, and/or the like). As should be understood, the sensing pads 130, 230, and/or support pads 250 may be independently secured into user-selected positions.

In various embodiments, the sensing pads 130, 230 are integrally formed and/or secured in a non-removeable manner to respective arms 120, 220. In various other embodiments, the sensing pads 130, 230 and/or support pads 250 are each configured to be clipped onto the respective one of the first arm 120A, 220A and the second arm 120B, 220B in a selected one of the first position and the second position via a corresponding clip 132 (see FIGS. 3 and 4) and/or pad bracket 260. For example, the first sensing pad 130A may be clipped to the first arm 120A in the first position and then unclipped from the first arm and reclipped to the first arm in the second position. Similarly, the first sensing pad 130A may be unclipped from being clipped to the first arm 120A in the second position and then reclipped to the first arm in the first position. The second sensing pad 130B may be similarly clipped, unclipped, and/or reclipped to the second arm 120B to switch the position of the second sensing pad 130B between the first position and the second position, or vice versa.

In various embodiments, the ability to move the sensing pads 130, 230 between the first position and second position (or vice versa) provides a technical improvement in the field of muscle testing. In particular, the number of different types of muscle tests that may be performed using the muscle testing device 100, 200 is greatly increased by the flexibility of the muscle testing device that is enabled by the ability to move the sensing pads 130, 230 between the first position and the second position (or vice versa).

Moreover, the ability of the muscle testing device 100, 200 to be operated without requiring a clinician assert a resistive force with their hand so as to hold a pressure or force sensor (e.g., a handheld myometer) in place while the patient applies a force thereto reduces the amount of noise in measurements and increases the accuracy of the measurements over conventional muscle testing techniques. Such features of the muscle testing device 100, 200 are enabled by the ability to use the muscle testing device 100, 200 bilaterally (e.g., by coupling two sensing pads 130, 230 thereto); ability to couple support pads 250 to the muscle testing device 100, 200 to aid in maintaining the patient's body and/or the muscle testing device 100, 200 in an appropriate position; and/or use of alignment mat 700, stand 800, table or plinth attachment 900, and/or the like to prevent the need for a clinician to hold the myometer in their hand.

FIG. 3 illustrates a sensing pad 130 having a sensing surface 135 built in, hard-wired, and/or the like into the sensing pad 130. For example, in various embodiments, a sensing pad 130 may comprise a sensor configured to measure pressure and/or force. For example, the sensing pad comprises a sensing surface 135. The sensing surface 135 comprises a smart material, force transducer(s), pressure sensors, and/or the like configured to measure force and/or pressure applied thereto.

In various embodiments, the first and second sensing pad 130A, 130B each comprise a sensing component configured to generate a signal corresponding to and/or measure a force and/or pressure applied thereto. For example, the sensing pads 130A, 130B may each comprise a force transducer and/or other force or pressure sensor. In further embodiments, a strain gauge may be provided to measure the force applied to the frame 105 (e.g., the central frame element 110) via the sensing pads 130A, 130B.

In various embodiments, the muscle testing device 100, 200 further comprises one or more user interfaces 140A, 140B. For example, the one or more user interfaces 140A, 140B may be configured to visually display and/or audibly provide a value of a force and/or pressure measured by one or more of the sensing pads 130A, 130B. For example, in an example, embodiment, a first user interface 140 is disposed on the first sensing pad 130A and provides a value of a force and/or pressure measured by the first sensing pad 130A and a second user interface 140 is disposed on the second sensing pad 130B and provides a value of a force and/or pressure measured by the second sensing pad 130B. In various embodiments, one or more user interfaces 140 may be disposed on the first and/or second arms 120A, 120B or the central frame element 110. For example, the user interface(s) 140 may be in communication with the sensing surface 135 of the first and/or second sensing pad 130A, 130B such that the user interface(s) 140 receives a measurement and/or signal from the sensing component of the first and/or second sensing pad 130A, 130B and may display and/or audibly provide a value indicating a force and/or pressure based on the received measurement and/or signal.

In various embodiments, the muscle testing device 100, 200 comprises a communications interface 150. In various embodiments, the communications interface 150 is configured to enable the muscle testing device 100, 200 to communicate with a computing device (e.g., a mobile phone, tablet, laptop, desktop computer, and/or other computing entity) via a wired and/or wireless communication protocol. In an example embodiment, the communications interface 150 is configured to enable the muscle testing device 100, 200 to communicate with a computing device via a network (e.g., local area network, wide area network, internet, and/or the like). For example, the communications interface 150 may enable the muscle testing device 100, 200 to be coupled to a computing device via a USB cord (or other wired connection) such that the computing device may receive signals indicating measurements corresponding to forces and/or pressures applied to the sensing surface(s) 135 of a respective the sensing pad(s) 130, 230. For example, the communications interface 150 may enable the muscle testing device 100, 200 to communicate with the computing device such that the computing device may receive signals indicating measurements corresponding to forces and/or pressures applied to the sensing surface(s) 135 of a respective sensing pad(s) 130, 230 via radio and/or infrared frequency communications (e.g., Bluetooth, Bluetooth lite, near field communication, Wi-Fi, and/or the like).

In various embodiments, a sensing pad 130, 230 may be configured to couple a myometer therein such that the myometer may be secured to the frame 105, 205. FIG. 4 illustrates an example embodiment of a sensing pad 130′ configured to couple a sensor device 400 (e.g., a pressure and/or force sensor) therein. In an example embodiment, the sensing pad 130′ is configured to secure a sensor device 400 coupled therein to an arm 120, 220 of the frame 105, 205 via a clip 132 or a pad bracket 260.

In various embodiments, the sensing pad 130′ comprises a cradle 134 configured to couple a sensor device 400 into the sensing pad 130′. For example, the cradle 134 may comprise a sensor receptacle 136 configured to couple a removeable sensor device 400 therein. For example, the sensor device 400 may be a handheld myometer or other force and/or pressure sensor. For example, the sensor device 400 may have a sensing surface 435 comprising smart material, force transducer(s), pressure sensors, and/or the like configured to measure force and/or pressure applied thereto. In various embodiments, the cradle 134 comprises a locking mechanism configured to lock a sensor device 400 to and/or into the sensor receptacle 136 of the cradle 134. For example, the sensor device 400 may rotate and/or slide into the sensor receptacle 136 of a cradle 134 to be secured and/or locked into the sensing pad 130′. When the sensor device 400 is coupled into the cradle 134, 240, the sensing pad 130′, 230 comprises a sensing surface.

In various embodiments, the sensor device 400 may have an integrated user interface 440 thereon. In an example embodiment, the sensor device 400 may electronically couple to the muscle testing device 100, 200 such that measurements captured by the sensor device 400 may be displayed and/or audibly provided on a user interface 140 integrated into the muscle testing device frame 105, 205 and/or provided (e.g., transmitted) via a communications interface 150 integrated into the muscle testing device frame 105, 205.

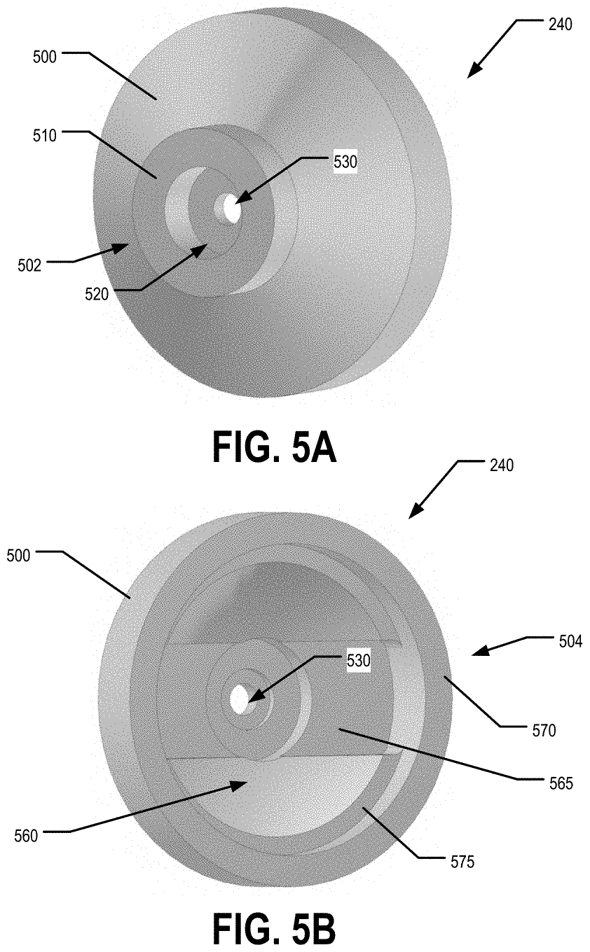

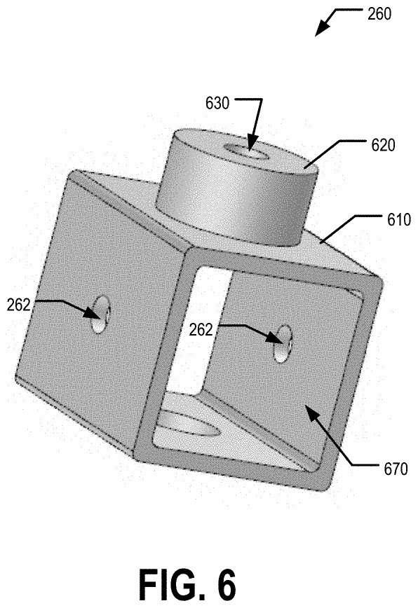

FIG. 5A provides a back perspective view of an example cradle 240, in accordance with an example embodiment, and FIG. 5B provides a front perspective view of the example cradle 240 illustrated in FIG. 5A. The example cradle 240 comprises a housing 500. The housing 500 comprises a bracket attachment mechanism 502 substantially disposed on a first side of the housing 500 and a sensor device attachment mechanism 504 substantially disposed on a second side of the housing 500 that is opposite the first side of the housing 500. FIG. 6 illustrates an example pad bracket 260 that may be used to couple the example cradle 240 to an arm 220 of a muscle testing device 100, 200, in an example embodiment.

In the illustrated embodiment, the bracket attachment mechanism 502 comprises a wall 510 that at least partially defines a bracket recess 520. In an example embodiment, the bracket attachment mechanism 502 further comprises through hole 530. In an example embodiment, the bracket recess 520 is configured to receive a protrusion 620 of a pad bracket 260 therein. For example, the cradle 240 may be coupled to the pad bracket 260 by positioning the protrusion 620 into the bracket recess 520 and securing a mechanical fastener through through hole 530 and through hole 630 of the pad bracket 260. The cradle 240 may then be secured and/or coupled to an arm 220 of the muscle testing device 200 by inserting the arm 220 into the opening 670 of the body 610 of the pad bracket 260 (such that the pad bracket 260 is configured to face the sensing device 400 in a desired one of the first position or the second position) and securing the pad bracket 260 to a particular predefined pad attachment location 222 by inserting a pin or other mechanical fastener 264 through the bracket attachment mechanism 262 and the particular predefined pad attachment location 222.

While FIG. 6 illustrates the opening 670 in the body 610 of the pad bracket 260 being square or rectangular in cross-section, it should be understood that the opening 670 may have other cross-sectional shapes in various embodiments. For example, in an example embodiment, the arms 220 may be circular in cross-sectional shape and the opening 670 may also be circular in cross-sectional shape such that a sensing pad 130, 230 may be moved between the first position and the second position without removing the pad bracket 260 from the arm 220. For example, in an example embodiment, the arms 220 may be circular in cross-sectional shape and the opening 670 may also be circular in cross-sectional shape such that a sensing pad 130, 230 may be rotated around a respective axis extending along the respective arm 120, 220 to a user-selected angle and/or position (with an appropriate level of precision for the application) without removing the pad bracket 260 from the arm 220.

In an example embodiment, the wall 510, bracket recess 520, and protrusion 620 are configured such that, when a sensor device 400 is secured to the cradle 240, the sensing surface 435 of the sensor device 400 is maintained in a particular plane. For example, the wall 510, bracket recess 520, and protrusion 620 are configured, in an example embodiment, such that, when a sensor device 400 is secured to the cradle 240, the sensing surface 435 of the sensor device 400 is maintained in a plane that is substantially perpendicular to the axis defined by the central frame element 110, 210. In an example embodiment, the wall 510, bracket recess 520, and protrusion 620 are configured such that, when a sensor device 400 is secured to the cradle 240, the sensor device 400 is rotatable about an axis that is substantially parallel to the axis defined by the central frame element 110, 210. In an example embodiment, the pad bracket 260 comprises the wall and bracket recess and the cradle 240 comprises the protrusion.

In various embodiments, the cradle 240 further comprises a sensor device attachment mechanism 504. In the illustrated embodiment, the sensor device attachment mechanism 504 comprises a sensor receptacle 560. In the illustrated embodiment, the sensor receptacle 560 is defined at least in part by peripheral barrier 570 and comprises seating 575 and groove 565. In various embodiments, the sensor receptacle 560 is configured to receive at least a portion of a sensor device 400 (e.g., a handheld myometer). In an example embodiment, the peripheral barrier 570 comprises one or more locking mechanisms configured to lock and retain the sensor device 400 within the sensor receptable 560. For example, in an example embodiment, the interior surface of the peripheral barrier 570 comprises threads or another mating mechanism configured to mate with and/or couple to corresponding threads or another mating mechanism of the sensor device 400. In an example embodiment, the interior surface of the peripheral barrier 570 comprises a frictional ring or coating (e.g., a rubbery ring or coating) configured to cause a sensor device 400 to be retained at least partially within the sensor receptacle 560 via friction.

In an example embodiment, the groove 565 is configured to receive a handle or hand strap of the sensor device 400 therein. For example, the seating 575 is configured to have at least a portion of a surface of the housing of the sensor device 400 rest there against. In an example scenario where the sensor device 400 comprises a handle or hand strap, the handle or hand strap may interfere with proper seating of the housing of the sensor device 400 against the seating 575. Thus, the groove 560 is configured to receive the handle or hand strap therein to prevent the handle or hand strap from interfering with the seating of the sensor device 400 within the sensor receptacle 560, in an example embodiment.

In various embodiments, the sensor device 400 comprises a built in or integral user interface (e.g., a display). In various embodiments, the sensor device attachment mechanism 504 is configured such that the user interface is viewable when the sensor device 400 is secured at least partially within the sensor receptacle 560. In an example embodiment, the cradle 240 is configured such that the sensor device 400 may be rotated about an axis that is substantially parallel to the axis defined by the central frame element 210. For example, this may enable a clinician or other user to view the display and read the measurement output displayed thereon (e.g., by rotating the sensor device 400) even when the display was not visible while the corresponding measurement was performed. In an example embodiment, the rotation of the sensor device 400 is enabled by the sensor receptacle 560. For example, in an example embodiment, the sensor device 400 is rotated with respect to the cradle 240. In an example embodiment, the rotation of the sensor device 400 is enabled by the coupling of the cradle 240 to the pad bracket 260. For example, the sensor device 400 and the cradle 240 may be rotatable with respect to the pad bracket 260. In various embodiments, the rotatability of the sensor device 400 while the sensor device 400 is coupled to the muscle testing device frame provides an improved ease of usability.

### Example Muscle Testing Kit

In various embodiments, a muscle testing kit is provided. In various embodiments a muscle testing kit comprises at least one muscle testing device 100, 200. In various embodiments, a muscle testing kit may comprise multiple muscle testing devices 100, 200 in various sizes. For example, muscle testing devices 100, 200 may be sized for use on one of infants/young children, older children/teenagers, adults, large adults, and/or the like.

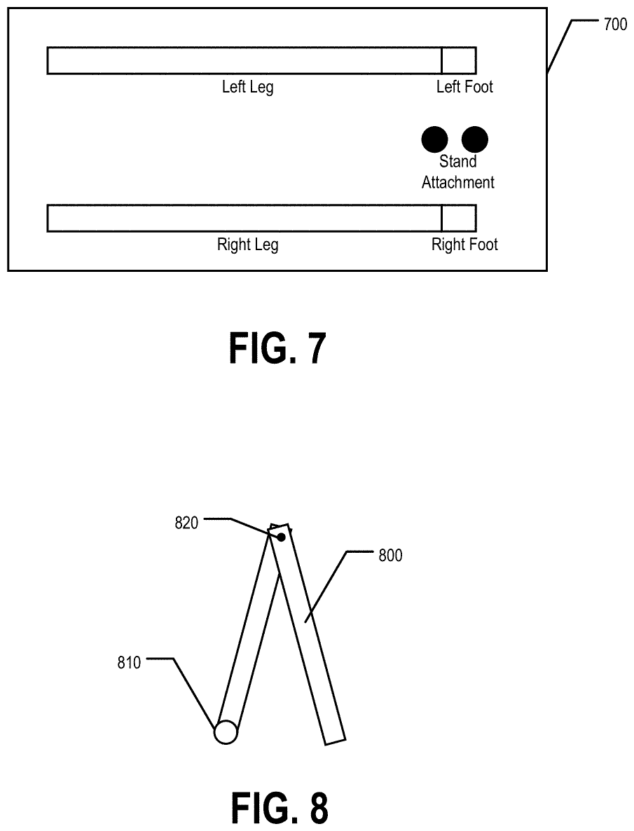

In various embodiments, a muscle testing kit comprises, in addition to at least one muscle testing device 100, 200, one or more of an alignment mat, stand, table or plinth attachment, support pads, a grip attachment, and/or range of motion device. FIG. 7 illustrates an example alignment mat 700. For example, the alignment mat 700 may be a flexible mat (e.g., foam, flexible plastic, and/or the like) and/or rigid mat (e.g., made of wood, rigid plastic, metal, and/or the like. The alignment mat 700 comprises markings along which a person's bones, limbs, joints, and/or the like may be aligned such that the person's body is in the appropriate position for the performance of one or more muscle strength tests. In various embodiments, the alignment mat 700 comprises markings indicating where a muscle testing device 100, 200 should be positioned for the performance of one or more muscle strength tests. In various embodiments, the alignment mat 700 comprises markings indicating where a stand to which the muscle testing device 100, 200 may be secured should be positioned for the performance of one or more muscle strength tests. In an example embodiment, the alignment mat 700 is a jig board and may comprise slots, notches, and/or the like configured for receiving a corresponding mating member disposed on a stand, such that the stand may be securely positioned on the alignment mat 700 in an appropriate position for the performance of one or more muscle strength tests. In various embodiments, the alignment mat 700 may comprise markings corresponding to a plurality of muscle strength tests. In various embodiments, the muscle testing kit comprises multiple alignment mats 700 with each alignment mat sized for use on one of infants/young children, older children/teenagers, adults, large adults, and/or the like.

FIG. 8 illustrates an example stand 800. In various embodiments, the stand 800 may comprise one or more supporting members configured to have a muscle testing device 100, 200 secured thereto such that the muscle testing device 100, 200 secured to the stand 800 is free standing. For example, the muscle testing device 100, 200 may be secured to the stand 800 via securing mechanism 810 such that the muscle testing device 100, 200 is free standing with assistance of the stand 800. In various embodiments, the stand 800 is configured to hold a muscle testing device 100, 200 secured thereto in an appropriate position for performance of one or more muscle strength tests. In an example embodiment, the stand 800 comprises one or more mating members configured to mate with corresponding mating members, notches, slots, and/or the like of an alignment mat 700 such that the stand 800 is held in appropriate position such that a muscle testing device 100, 200 secured to the stand 800 is positioned for performance of one or more muscle strength tests. In an example embodiment, the stand 800 comprises one or more wheels 810 such that the stand (possibly having the muscle testing device 100, 200 secured thereto) may be wheeled (e.g., tilted and wheeled) over to a patient in a chair or seating on a table or plinth.

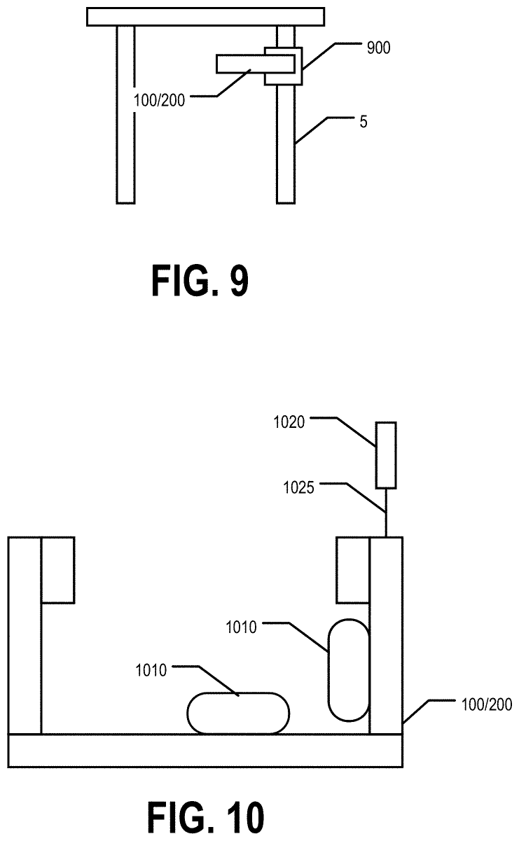

FIG. 9 illustrates an example table or plinth attachment 900 configured to couple a muscle testing device 100, 200 to a table or plinth 5. For example, the table or plinth attachment 900 may be configured to be secured to a table (e.g., table top, table leg, and/or the like) or a plinth. The table or plinth attachment 900 may further be configured to have a muscle testing device 100, 200 secured thereto. For example, the table or plinth attachment 900 may be configured to secure the muscle testing device 100, 200 in a position where the muscle testing device 100, 200 may be used to perform a knee flexion, knee extension, and/or other muscle strength test.

FIG. 10 illustrates an example muscle testing device 100, 200 having one or more support pads 1010 secured thereto and having a grip attachment 1020 secured thereto. In various embodiments, the support pads 1010 may be attached and/or removed from the central frame element 110/210 and/or arm 120A/220A, 120B/220B. For example, the support pads 1010 may be configured to assist in the positioning of a person's body and/or of the muscle testing device 100, 200 with respect to the person's body in an appropriate position for performance of one or more muscle strength tests. For example, the support pads 1010 may be configured to hold a person's elbow at 90 degrees for the performance of a grip strength test using the grip attachment 1020. In various embodiments, the grip attachment 1020 may be coupled to the electronics of the muscle testing device 100, 200 via cable or cord 1025. In various embodiments, the grip attachment 1020 is configured to measure a squeezing force or pressure applied thereto. The cable or cord 1025 may provide a signal generated by one or more sensors of the grip attachment 1020 to circuitry of the muscle testing device 100, 200 such that a value representative of the squeezing force or pressure applied to the grip attachment 1020 is displayed via the user interface 140A, 140B of the muscle testing device 100, 200 or transmitted and/or provided via the communications interface 150 of the muscle testing device 100, 200. In an example embodiment, the muscle testing device 100, 200 is used to hold the patient's body in the appropriate position and the grip attachment 1020 is not electrically coupled to the muscle testing device 100, 200. In such an embodiment, the value representative of the squeezing force or pressure applied to the grip attachment 1020 is displayed via a user interface of the grip attachment 1020.

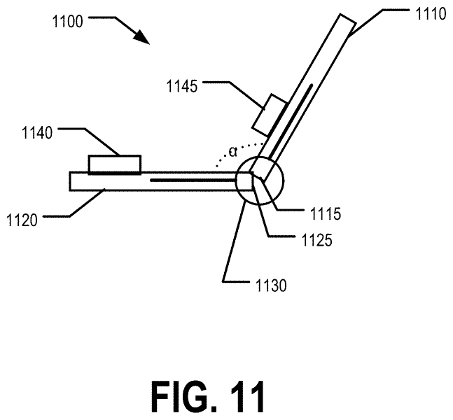

In an example embodiment, the muscle testing kit may comprise a range of motion device, such as an ankle range of motion device 1100 shown in FIG. 11.

### Example Range of Motion Device

FIG. 11 illustrates an example ankle range of motion device 1100. In various embodiments, the range of motion device 1100 may be used to accurately measure the range of motion of a human ankle or other non-hinge joint in a reproducible manner. In various embodiments, the range of motion device 1100 comprises a first planar member 1110 and a second planar member 1120. In various embodiments, the first planar member 1110 and/or the second planar member 1120 may be a plastic, wooden, and/or metal plate. The first planar member 1110 comprises a first surface 1115, which is an edge of the plate of the first planar member 1110. The second planar member 1120 comprises a second surface 1125, which is an edge of the plate of the second planar member 1120. In various embodiments, the first surface 1115 of the first planar member 1110 is secured to the second surface 1125 of the second planar member 1120 in a hinged manner. For example, the first planar member 1110 and the second planar member 1120 may be in fixed relationship with one another other than the angle α between the first planar member 1110 and the second planar member 1120. For example, the angle α is changeable. In various embodiments, the range of motion device 1100 comprises a goniometer 1130 configured to measure the angle α.

In various embodiments, the range of motion device 1100 may further comprise straps 1140, 1145. For example, the range of motion device 1100 may comprise one or more first straps 1145 configured to strap or temporarily secure a portion of a human body to the first planar member 1110. For example, the one or more first straps 1145 may be configured to strap a human foot to the first planar member 1110. For example, the range of motion device 1100 may comprise one or more second straps 1140 configured to strap or temporarily secure a portion of a human body to the second planar member 1120. For example, the one or more second straps 1140 may be configured to strap a human leg (e.g., lower leg) to the second planar member 1120.

As will be appreciated from the description herein, various embodiments of the muscle testing kit are configured to enable muscle testing measurements of one or more of: ankle dorsiflexion, ankle plantarflexion, elbow flexion, elbow extension, functional elbow flexion, functional elbow extension, knee flexion, knee extension, hip abduction, hip adduction, hip extension supine, hip abduction clamshell, shoulder abduction, shoulder adduction, shoulder horizontal adduction, shoulder internal rotation, shoulder external rotation, grip strength, neck lateral flexion, neck extension, and neck forward flexion.

### CONCLUSION

Additional information regarding various embodiments may be found in the attached Appendix. Many modifications and other embodiments will come to mind to one skilled in the art to which this disclosure pertains having the benefit of the teachings presented in the foregoing descriptions and the associated drawings. Therefore, it is to be understood that embodiments are not to be limited to the specific embodiments disclosed and that modifications and other embodiments are intended to be included within the scope of the appended claims. Although specific terms are employed herein, they are used in a generic and descriptive sense only and not for purposes of limitation.

1. A muscle testing device comprising:
a frame comprising a central frame element and two arms extending from the central frame element, wherein a distance between the two arms is adjustable; and
a first sensing pad (a) configured to be secured to a first arm of the two arms, (b) configured to measure a force or pressure applied to a sensing surface thereof, and (c) being moveable between a first position about the first arm and a second position about the first arm.

2. The muscle testing device of claim 1, wherein when the first sensing pad is secured to the first arm in the first position, the sensing surface of the first sensing pad faces a second arm of the two arms and when the first sensing pad is secured to the first arm in the second position, the sensing surface of the first sensing pad faces away from the second arm.

3. The muscle testing device of claim 1, wherein the first sensing pad is moveable between the first position and the second position by rotating a pad bracket configured to couple the at least one sensor pad to the first arm with respect to the first arm.

4. The muscle testing device of claim 1, wherein the first sensing pad comprises a cradle and a sensor device, the cradle is coupled to the first arm by a pad bracket, and the cradle is configured to couple the sensor device to the first arm, the sensor device comprising the sensing surface.

5. The muscle testing device of claim 4, wherein the pad bracket is configured to couple the cradle to the first arm at a selected position along the first arm such that the first arm has a selected effective length.

6. The muscle testing device of claim 4, wherein one of:
the cradle and the pad bracket are configured such that the cradle is rotatable with respect to the pad bracket, while the cradle is coupled to the pad bracket, about an axis that is transverse to a direction substantially parallel to the first arm; or
the cradle and the sensor device are configured such that the sensor device is rotatable with respect to the cradle, while the sensor device is coupled to the cradle, about the axis that is transverse to the direction substantially parallel to the first arm.

7. The muscle testing device of claim 4, wherein the sensor device is a myometer.

8. The muscle testing device of claim 1, further comprising at least one user interface configured to provide human perceivable output of a muscle strength measurement.

9. The muscle testing device of claim 8, wherein the user interface is integrated into the at least one sensing pad.

10. The muscle testing device of claim 1, further comprising a pressure and/or force sensor integrated into the at least one sensing pad and comprising the sensing surface, the pressure and/or force sensor configured to measure pressure and/or force applied to the sensing surface thereof.

11. The muscle testing device of claim 1, wherein the first arm and second arm are respectively movable between respective in-use positions where a respective arm of the first arm and the second arm extends in a direction that is transverse to a direction substantially parallel to the central frame element and respective stowed positions where the respective arm extends in a direction that is substantially parallel to the central frame element.

12. The muscle testing device of claim 1, further comprising a second sensing pad or a support pad coupled to the second arm.

13. A muscle testing kit comprising:
at least one of a muscle testing device or a muscle testing device frame,
wherein the muscle testing device comprises:
a frame comprising a central frame element and two arms extending from the central frame element, wherein a distance between the two arms is adjustable; and
a first sensing pad (a) configured to be secured to a first arm of the two arms, (b) configured to measure a force or pressure applied to a sensing surface thereof, and (c) being moveable between a first position and a second position, wherein when the first sensing pad is secured to the first arm in the first position, the sensing surface of the first sensing pad faces a second arm of the two arms and when the first sensing pad is secured to the first arm in the second position, the sensing surface of the first sensing pad faces away from the second arm, and wherein the muscle testing device frame comprises:
the frame comprising the central frame element and the two arms extending from the central frame element, wherein the distance between the two arms is adjustable; and
a cradle coupled to the first arm of the two arms via a pad bracket and configured to couple a sensor device to the first arm, wherein when the cradle is secured to the first arm in the first position, a sensing surface of the sensor device coupled to the cradle faces the second arm and when the cradle is secured to the first arm in the second position, the sensing surface of the sensor device coupled to the cradle faces away from the second arm.

14. The muscle testing kit of claim 13 further comprising a stand, the at least one of the muscle testing device or the muscle testing device frame configured to be coupled to the stand and the stand configured to maintain the at least one of the muscle testing device or the muscle testing device frame in one or more designated configurations when the at least one of the muscle testing device or the muscle testing device frame is coupled to the stand.

15. The muscle testing kit of claim 13 further comprising at least one of:
an alignment mat illustrating positioning of one or more limbs of a patient for one or more muscle testing measurements,
a table or plinth attachment configured to secure the at least one of the muscle testing device or the muscle testing device frame to a table or plinth, or
a grip attachment configured to measure grip strength.

16. The muscle testing kit of claim 15, wherein the alignment mat is a jig board configured to have the at least one of the muscle testing device or the muscle testing device frame coupled thereto in one or more designated configurations for the one or more muscle testing measurements.

17. The muscle testing kit of claim 13 further comprising one or more support pads configured to be selectively secured to the at least one of the muscle testing device or the muscle testing device frame and to be positioned against a patient's body to properly position the patient's body for one or more muscle testing measurements.

18. The muscle testing kit of claim 13, wherein the muscle testing kit is configured to enable muscle testing measurements of one or more of ankle dorsiflexion, ankle plantarflexion, elbow flexion, elbow extension, functional elbow flexion, functional elbow extension, knee flexion, knee extension, hip abduction, hip adduction, hip extension supine, hip abduction clamshell, shoulder abduction, shoulder adduction, shoulder horizontal adduction, shoulder internal rotation, shoulder external rotation, grip strength, neck lateral flexion, neck extension, and neck forward flexion.

19. An ankle range of motion measuring device, comprising:
a first planar member;
a second planar member; and
a goniometer,
wherein the first planar member comprises a first surface, the second planar member comprises a second surface, the first surface of the first planar member is coupled to the second surface of the second planar member in a hinged manner, and the goniometer is coupled to the first and second planar members such that the goniometer is configured to measure an angle between the first planar member and the second planar member.

20. The angle range of motion measuring device of claim 19 further comprising:
a first strap configured to strap a human foot to the first planar member; and
a second strap configured to strap a human leg to the second planar member.
